(19) United States
(12) Patent Application Publication
(10) Pub. No.: US 2018/0207034 A1
(43) Pub. Date: Jul. 26, 2018

(54) ELECTRICALLY CONDUCTIVE BANDAGE FOR USE WITH TOUCHSCREEN DEVICES

(71) Applicant: Dimitrije Stojanovski, Shelby Township, MI (US)

(72) Inventor: Dimitrije Stojanovski, Shelby Township, MI (US)

(21) Appl. No.: 15/842,170

(22) Filed: Dec. 14, 2017

Related U.S. Application Data
(63) Continuation-in-part of application No. 14/989,186, filed on Jan. 6, 2016.
Application No. 14/989,186 is a continuation-in-part of application No. 14/190,220, filed on Feb. 26, 2014, now abandoned.
(60) Provisional application No. 62/533,887, filed on Jul. 18, 2017.
(60) Provisional application No. 62/555,264, filed on Sep. 7, 2017.

(51) Int. Cl. A61F 13/00; A61F 13/02; G06F 3/0354; G06F 3/041

(52) U.S. Cl. CPC A61F 13/00051; A61F 13/0206; A61F 13/0223; G06F 2203/0331; A61F 13/0253; G06F 3/03545; G06F 3/041; A61F 13/0226

(57) ABSTRACT

A bandage covers skin of a patient for the purpose of fostering healing. A touchscreen device normally utilizes conductivity of skin to sense location of a finger upon the touchscreen device. A bandage is includes adhesive configured to attach the bandage to the skin and a polymer-based bandage surface providing electrical conductivity between a plurality of locations on a surface of the bandage, the polymer-based bandage surface comprising one of an anti-static conductive polymer adhesive, a conductive silicone rubber, and a conductive foam material.

### CROSS REFERENCE TO RELATED APPLICATIONS

This disclosure is a continuation-in-part application of U.S. application Ser. No. 14/989,186 filed on Jan. 6, 2016 which is a continuation-in-part application of U.S. application Ser. No. 14/190,220 filed on Feb. 26, 2014; claims priority from U.S. Provisional Application No. 62/533,887 filed on Jul. 18, 2017; and claims priority from U.S. Provisional Application No. 62/555,264 filed on Sep. 7, 2017 which are all hereby incorporated by reference.

### FIELD OF THE DISCLOSURE

The present disclosure relates generally to bandages for protection of injured skin. In particular, examples of the present disclosure are related to bandages manufactured with conductive material for use with touchscreen devices.

### BACKGROUND

The statements in this section merely provide background information related to the present disclosure. Accordingly, such statements are not considered to constitute an admission of prior art.

A bandage is a strip of material used to protect, immobilize, compress or support a wound or injured body part. Bandages are available in a wide range of types, from cloth strips to specialized shaped bandages designed for specific body parts or types of injuries. Dressings are materials that are applied directly to wounds to promote healing and prevent further harm to the site of injury.

Typical bandages found in typical home first-aid kits are strips made of plastic, fabric or other suitable materials, with an adhesive side which is placed on the skin and an absorbent pad adhered on the adhesive side which is placed directly over the injured skin. Typical absorbent pads are made out of cotton, polyester or other suitable materials. Other bandages consist of strips of material alone, which do not adhere to the skin but cohere to themselves, for use with separate absorbent pads.

Touchscreen devices employ electronic visual displays that the user can control by touching the screen with a finger or other object such as a stylus. Touchscreens are common in devices such as tablet computers and smartphones. Many touchscreens employ technology that requires an electrically conductive object to touch the screen in order for the user to be able to use the touchscreen device. Human skin is electrically conductive, and can be used to interact with touchscreen devices.

However, certain circumstances arise in which skin must be kept covered. For example, when skin is injured, it is recommended that the skin be kept covered with a bandage. In such circumstances, the wound covering prevents electrically conductive skin from coming into direct contact with touchscreen devices that employ conductive technology, and therefore touchscreen devices can be used only with difficulty when skin must remain covered.

As they are currently manufactured, typical bandages cannot be used with touchscreen devices, as they lack the electrically conductive properties to do so.

Studies have been performed on silver containing dressings. Some studies have shown improvements in wound healing times and healthful results when silver is in contact with a wound site during the healing process.

### SUMMARY

A bandage covers skin of a patient for the purpose of fostering healing. A touchscreen device normally utilizes conductivity of skin to sense location of a finger upon the touchscreen device. A bandage is includes adhesive configured to attach the bandage to the skin and a polymer-based bandage surface providing electrical conductivity between a plurality of locations on a surface of the bandage, the polymer-based bandage surface comprising one of an anti-static conductive polymer adhesive, a conductive silicone rubber, and a conductive foam material.

### BRIEF DESCRIPTION OF THE DRAWINGS

Non-limiting and non-exhaustive embodiments of the present disclosure are described with reference to the following figures, wherein like reference numerals refer to like parts throughout the various views unless otherwise specified.

FIGS. 1A and 1B illustrate exemplary designs of conductive cloth for use in bandages that can be used with touchscreen devices, in accordance with the present disclosure;

FIG. 1A shows an exemplary woven design of conductive material utilizing straight threads; and

FIG. 1B shows an exemplary woven design of conductive material utilizing metallic threads woven at angles;

FIGS. 2A and 2B illustrate a top and side view of a bandage manufactured using conductive material, in accordance with the present disclosure;

FIG. 2C illustrates a side view of a bandage manufactured using a combination of conductive and non-conductive layers of material, in accordance with the present disclosure;

FIG. 2D shows a side view of bandage manufactured using a combination of conductive and non-conductive materials, wherein non-conductive material is exposed to the touchscreen surface, in accordance with the present disclosure;

FIG. 2E shows a side view of bandage manufactured using a combination of conductive and non-conductive materials, a conductive layer wraps around at least one side of the bandage, in accordance with the present disclosure;

FIG. 2F illustrates a plurality of conductive material patterns that can be provided upon a bandage, in accordance with the present disclosure;

FIG. 3 illustrates a finger wrapped with a bandage configured to wrap around a part of a patient and cohere to itself, the bandage including conductive properties, in accordance with the present disclosure;

FIG. 4 shows an exemplary bandage with conductive properties, manufactured by spraying conductive material directly onto an outside face of bandage, in accordance with the present disclosure;

FIG. 5 illustrates an exemplary bandage, wherein conductive material in the bandage permits an electrical circuit to be created through the finger of the wearer, in accordance with the present disclosure;

FIG. 6 illustrates through a photographic image a design for a bandage including metallic material in the cloth portion of the bandage, wherein the metallic material is spaced evenly throughout the cloth fibers and is present is sufficient quantity to provide a metallic appearance to the cloth portion of the bandage, in accordance with the present disclosure;

FIG. 7 illustrates through a photographic image the design of FIG. 6, in accordance with the present disclosure;

FIG. 8 illustrates through a photographic image alternate configurations of the design of FIG. 6, the configurations being part of the same single design as illustrated in FIG. 6, in accordance with the present disclosure; and

FIG. 9 illustrates through a photographic image alternate configurations of the design of FIG. 6, the configurations being part of the same single design as illustrated in FIG. 6, in accordance with the present disclosure.

Corresponding reference characters indicate corresponding components throughout the several views of the drawings. Skilled artisans will appreciate that elements in the figures are illustrated for simplicity and clarity and have not necessarily been drawn to scale. For example, the dimensions of some of the elements in the figures may be exaggerated relative to other elements to help to improve understanding of various embodiments of the present disclosure. Also, common but well-understood elements that are useful or necessary in a commercially feasible embodiment are often not illustrated in order to facilitate a less obstructed view of these various embodiments of the present disclosure.

### DETAILED DESCRIPTION

In the following description, numerous specific details are set forth in order to provide a thorough understanding of the present disclosure. It will be apparent, however, to one having ordinary skill in the art that the specific detail need not be employed to practice the present disclosure. In other instances, well-known materials or methods have not been described in detail in order to avoid obscuring the present disclosure.

Reference throughout this specification to “one embodiment”, “an embodiment”, “one example” or “an example” means that a particular feature, structure or characteristic described in connection with the embodiment or example is included in at least one embodiment of the present disclosure. Thus, appearances of the phrases “in one embodiment”, “in an embodiment”, “one example” or “an example” in various places throughout this specification are not necessarily all referring to the same embodiment or example. Furthermore, the particular features, structures or characteristics may be combined in any suitable combinations and/or sub-combinations in one or more embodiments or examples. In addition, it is appreciated that the figures provided herewith are for explanation purposes to persons ordinarily skilled in the art and that the drawings are not necessarily drawn to scale.

Embodiments in accordance with the present disclosure may be embodied as an apparatus or a method.

Metallic thread and other types of conductive materials are not as stretchable as cloth fabric. Conductive materials have been manufactured in a woven pattern that can stretch further than previously known conductive materials. Such patterns take advantage of bends in the threads making up the weaves to compensate for use of a rigid, unstretchable thread. Use of a woven pattern that enables a cloth to stretch is advantageous for embodiments that can benefit both from properties enabled by use of a metallic cloth and properties enabled by using a stretchable material. Retention of such electrically conductive properties is important, for example, for the manufacture of articles that can be used with touchscreen devices.

Metallic materials can be used to provide conductive properties to a bandage. In another embodiment, non-metallic or organic conductive materials can be utilized. An exemplary anti-static conductive polymer adhesive can be used to provide conductivity to a bandage. Similarly, an exemplary conductive silicone rubber or a conductive foam material can be used to provide conductivity. Such products are known in the art and will not be described in detail herein. Such materials need to be selected based upon properties permitting the conductive material to be in close proximity to the skin of a patient according to criteria known in the art related to health care products, such as non-toxicity. Many conductive materials can be utilized according to the disclosed device, and the disclosure is not intended to be limited to the particular examples provided herein.

In addition, metallic and other types of conductive material typically are not breathable, in that they create a barrier which does not allow air to reach the skin or substances to evaporate from the skin. Incorporating metallic and other types of conductive material can be accomplished in a way to allow for a resulting material that retains some breathability, which is important for maintaining healthy and comfortable skin, especially when the skin is wounded and must be kept covered by a bandage.

Typical bandages found in typical home first-aid kits are thin, flexible, stretchable strips of material that come in various shapes and sizes for use with different types of wounds or injuries on different body parts. In general, the bandage has an adhesive face, which contains an adhesive that allows the bandage to adhere to the skin and to itself. An absorbent pad to be placed directly over the site of injury is typically adhered to the adhesive side. This absorbent pad may be made of cotton, polyester, or any other suitable material. The non-adhesive, outside face of the bandage faces away from the skin.

Touchscreen devices are known in the art and will not be disclosed in detail herein. A touchscreen device is known to sense a location of a user's finger by sensing conduction of electricity from one location on the screen surface, through the finger of the user, and to a second location on the screen surface. In accordance with various embodiments of the present disclosure, an electrically conductive bandage for use with touchscreen devices is provided. A bandage includes conductive material and enables use of a touchscreen device by conducting electricity from one point on the touchscreen device to another point on the touchscreen device.

In some embodiments, the bandage is manufactured with only conductive materials, and any absorbent pad or dressing, whether separate or combined, is manufactured with conventional materials. In one example, such a bandage is manufactured with small holes for ventilation of the skin, to increase breathability for the comfort and health of the user. In other embodiments, any absorbent pad or other dressing combined with the abovementioned bandage also contains conductive material. In further embodiments, both the bandage and any absorbent pad or other dressing combined with the bandage are conductive.

In some embodiments, a layer of conductive material can be deposited or adhered to an outside surface of the bandage. Such a layer, for example, can include a metallic foil. In another example, the layer can include a sprayed on or brushed on layer of conductive material.

In some embodiments, the bandage is made conductive by manufacturing the bandage from a blend of conductive material and conventional materials. In one embodiment, a thread used to make a cloth bandage can include a composite of metallic fibers and conventional fibers, such as cotton or polyester. In another embodiment, a metallic thread can be used in a weave pattern with other non-conductive threads. In other embodiments, the bandage is made conductive by incorporating conductive material into the conventional material of the absorbent pad or other dressing combined with a conventional or conductive bandage. For example, very fine metallic threads can be blended into a cotton absorbent pad. In further embodiments, the bandage is made conductive by first combining conductive particles and adhesive into a mixture and then spraying such a mixture of conductive particles and adhesive directly onto the outside, non-adhesive face of the bandage.

A bandage can conduct electricity along a span of the bandage. Additionally or in the alternative, a bandage can conduct electricity from an outside surface to a contact point with the skin of the wearer in at least two places or points, and the electrical conductivity of the skin of the wearer can be used to complete a conductive circuit between conductive points on the bandage.

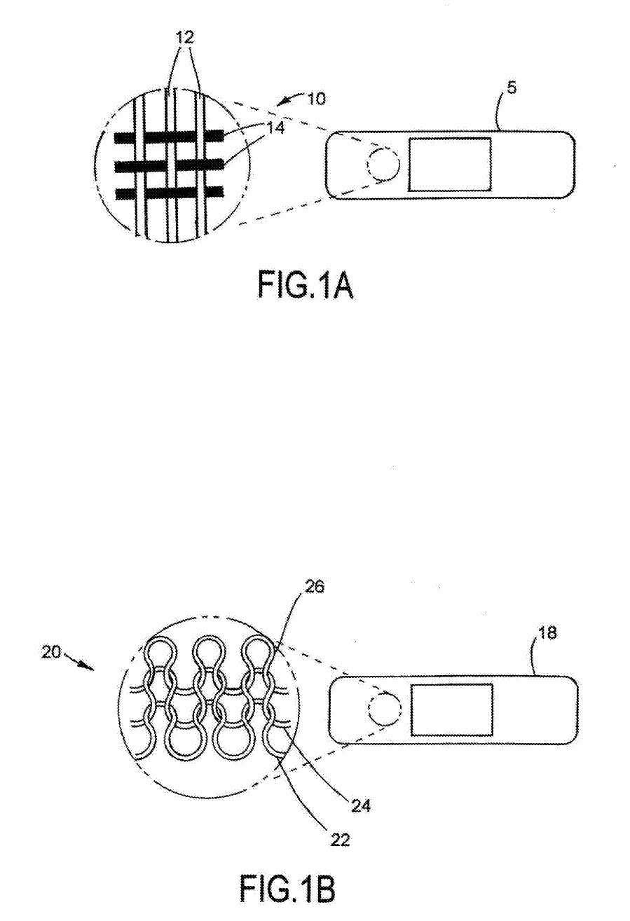

To illustrate, FIGS. 1A and 1B illustrate exemplary designs of conductive cloth for use in bandages that can be used with touchscreen devices.

FIG. 1A shows an exemplary woven design of conductive material utilizing straight threads. Bandage 5 is illustrated including a close up view of fabric 10. Fabric 10 is constructed including a plurality of threads 12 in one direction interlaced with a plurality of threads 14 in another direction, frequently at 90 degree angles to each other. The metallic threads are retained in a straight shape, with threads interwoven at 90 degree angles. In one embodiment, all threads are made conductive material. In other embodiments, some of the threads are made of conductive material and others are made of non-conductive material. Such a woven design is easily and inexpensively constructed. Such a fabric tends to only be elastic if the materials used in the threads are elastic. According to one embodiment, threads 12 can be made of an elastic non-conductive material, such that the fabric is elastic in the direction of those threads, and threads 14 can be made of conductive, inelastic threads providing the touchscreen functionality disclosed herein.

FIG. 1B shows an exemplary woven design of conductive material utilizing metallic threads woven at angles. Bandage 18 is illustrated including a close up view of fabric 20. Fabric 20 is illustrated including a plurality of interwoven threads 22, 24, and 26. In one embodiment, all threads are made conductive material. In other embodiments, some of the threads are made of conductive material and others are made of non-conductive material. Each of the threads bend at after looping with a neighboring thread. When the fabric is pulled in one direction, the threads can flex, giving the fabric stretchability. Fabric 20 permits use of conductive, inelastic threads in a woven fabric, wherein the fabric is elastic due to the construction of the weave.

A number of fabric configurations are known in the art and include a wide variety of thread patterns. A number of different fabric configurations are envisioned for use with the bandages disclosed herein, and the disclosure is not intended to be limited to the particular examples provided herein.

Further embodiments of the present disclosure include bandages made of materials that are manufactured using a combination of conventional non-conductive material that is used in typical non-conductive bandages and the conductive material shown in FIGS. 1A and 1B. For example, such blended material can be made of alternating conductive and non-conductive threads in a variety of patterns and types. Inclusion of non-conductive material would further increase stretchability and breathability of the bandage and can potentially reduce the material costs of the bandage. Stretchability is important for the fit of the bandage over and around the user's site of injury. Breathability is important for the skin surrounding the site of injury to remain healthy. Both stretchability and breathability in bandages can be important for proper healing.

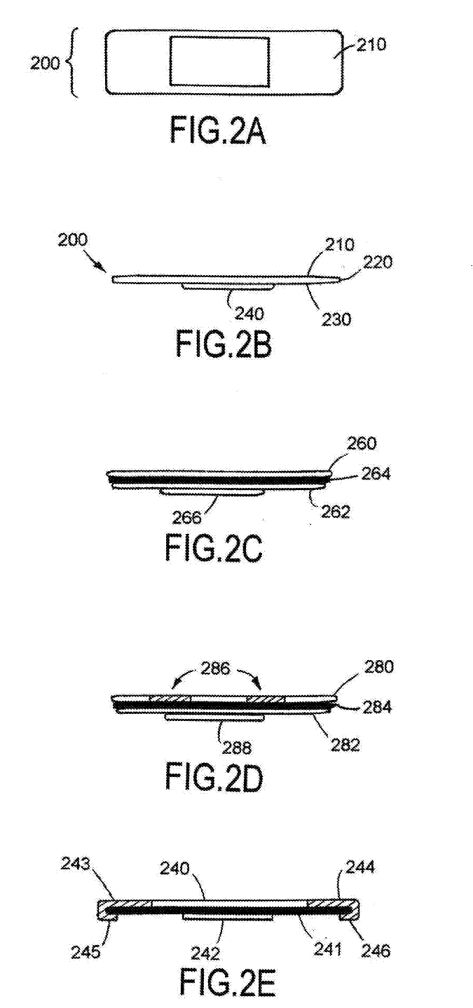

FIGS. 2A and 2B illustrate a top and side view of a bandage manufactured using conductive material, respectively. Bandage 200 has a non-adhesive side 210 that faces away from the skin of the user. FIG. 2B shows bandage 200 including non-adhesive side 210 and adhesive side 230, which adheres to the skin of the user. Absorbent pad 240 is placed over the injured skin of the user to absorb blood and other material, as well as protect the site of injury. Bandage 200 can be made solely of conductive material 230, such as foil. Bandage 200 can be made of a woven material including conductive threads disclosed herein. In some embodiments, the absorbent pad is made entirely of conventional materials, such as cotton. In some embodiments, the absorbent pad 240 may also be manufactured to contain conductive material.

Breathability is especially important in bandages made solely of conductive material, as metallic and other types of conductive material typically are not breathable. Breathability is important for the comfort of the user and the proper wound healing. Therefore, in some embodiments, bandages made solely of conductive materials may be manufactured with small holes or other openings to allow for greater breathability. In another embodiment, a thread density of a woven pattern can be modulated or selected to enhance breathability.

FIG. 2C illustrates a side view of a bandage manufactured using a combination of conductive and non-conductive layers of material. The conductive and non-conductive materials are not blended or woven together in this embodiment. Instead, in this embodiment, the conductive material 260 is adhered using adhesive material 264 to a typical bandage 262 made of conventional, non-conductive material. The adhesive can be any adhesive known in the art for use within a medical bandage. Typical bandage 262 can be made of cloth, plastic, rubber or other suitable, conventional, non-conductive material that will adhere to the adhesive on the inside face of the conductive material.

FIG. 2D shows a side view of bandage manufactured using a combination of conductive and non-conductive materials, wherein non-conductive material is exposed to the touchscreen surface. In this embodiment, the conductive material 284 is layered between two strips of conventional bandage material 280 and 282. In some embodiments, the conductive material may be layered in various different configurations. In further embodiments, the absorbent pad 288 may be manufactured to contain conductive material. A plurality of conductive zones 286 are illustrated in layer 280, permitting conduction of electricity through each of the zones 286 to the conductive material 284. An electrical circuit is created from a touchscreen surface proximate to one of the conductive zones 286, through the conductive zone 286, through conductive material 284, through the other, second conductive zone 286, and back to the touchscreen surface proximate to the second conductive zone 286. Conductive zone 286 can include some conductive threads interwoven with the material in that area. Conductive zone 286 can include holes with some of the conductive material 284 indented or formed to protrude through the holes. Conductive zone 286 can include an metallic or conductive ionic substance sprayed or brushed on the material.

FIG. 2E shows a side view of bandage manufactured using a combination of conductive and non-conductive materials, a conductive layer wraps around at least one side of the bandage. A bandage is illustrated including a first layer 240 and a second layer 241. An absorbent pad 242 is provided. Layers 240 and 241 can include conductive or non-conductive materials. Two portions of layer 240 include side portions 243 and 244 with conductive materials therein. Each of side portions 243 and 244 include wrap around sections 245 and 246, respectively. Conductivity can be provided or augmented by directly connecting portions 243 and 244 exposed to a phone screen surface to the skin of the patient at the respective wrap around sections. The wrap around sections can be used on one or more surfaces of the bandage to create or augment conductivity across the bandage. The wrap around sections can be threaded metallic fibers, spray or brush on materials, conductive polymers, or any other conductive material as disclosed herein.

FIG. 2F illustrates a plurality of conductive material patterns that can be provided upon a bandage. Bandage 290 includes a stripe 291 of conductive material running longitudinally along the bandage. Bandage 292 includes a cross-hatch pattern 293 of conductive material. Bandage 294 includes alternating bands of conductive material 295 and non-conductive material 296. The embodiments of FIG. 2F are provided as examples of patterns of conductive material that can be applied or integrated within a bandage. A number of patterns of conductive material are envisioned, and the disclosure is not intended to limited to the examples provided herein.

The exemplary configurations disclosed herein can be used with many types of bandages, for example, a wrap bandage typically used to hold absorbent pads or other material in place. This type of bandage does not adhere to the skin, but rather coheres to itself as it is wound around the injured body part and any absorbent material that has been placed on the skin.

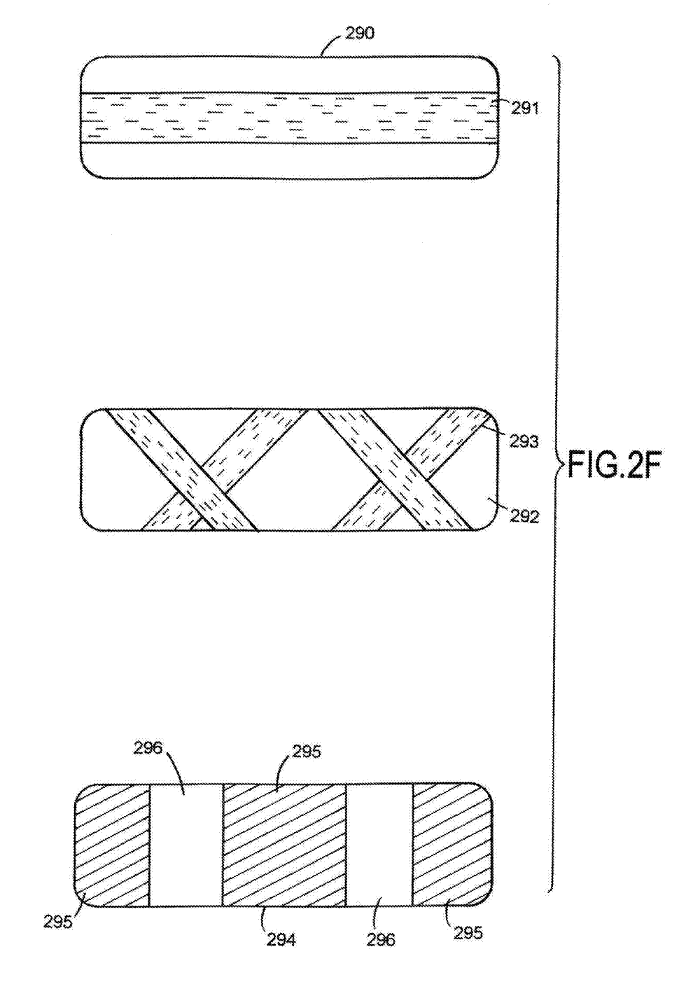

FIG. 3 illustrates a finger wrapped with a bandage configured to wrap around a part of a patient and cohere to itself, the bandage including conductive properties as disclosed herein. Configuration 300 includes wrap bandage 301 including conductive threads, a conductive layer, or other means of conductivity as disclosed herein. In the exemplary embodiment of FIG. 3, a finger 310 is encased within a splint device 320 known in the art including padding 322. A first wrap 330A and a second wrap 330B of bandage 301 are shown in cross-section. A similar wrap bandage can be used without the splint device. The wrap bandage 301 includes conductive properties such that finger 310 can be utilized to activate a touch screen device, as disclosed herein.

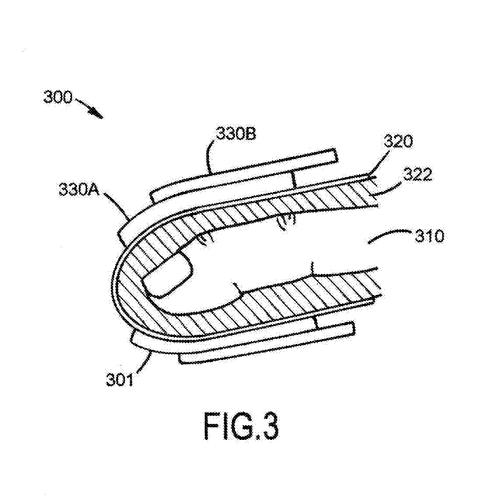

FIG. 4 shows an exemplary bandage with conductive properties, manufactured by spraying conductive material directly onto the outside face of bandage. Conductive particles can be mixed with an adhesive spray-able liquid. The adhesive material will allow the conductive particles to be permanently joined to the outside, non-adhesive face 410 of a conventional bandage. As conventional bandages are available in a variety of materials, including fabric, plastic and rubber, among others, various adhesive substances and resulting mixtures may be necessary. The resulting mixture 470 is then sprayed from exemplary spray can 460 directly on the outside, non-adhesive face 410 of a conventional bandage, which faces away from the skin of the user.

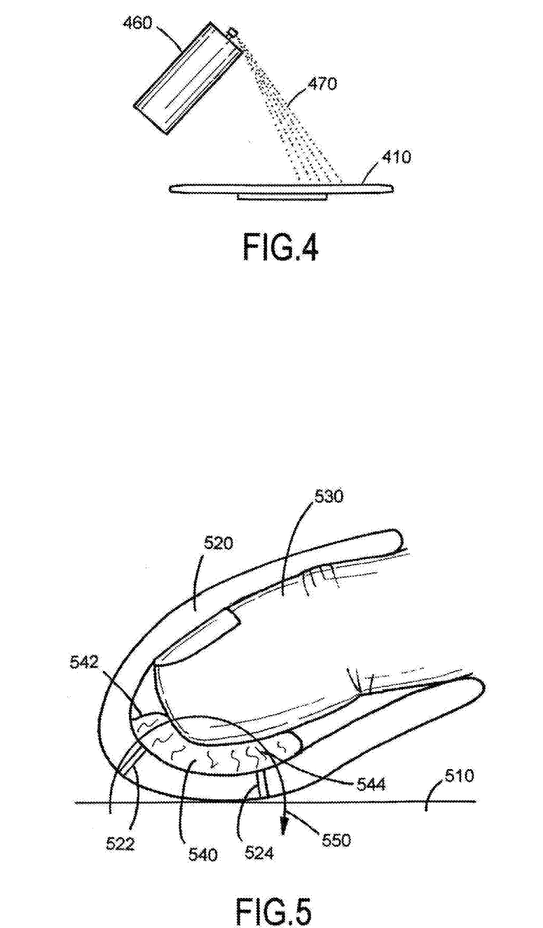

FIG. 5 illustrates an exemplary bandage, wherein conductive material in the bandage permits an electrical circuit to be created through the finger of the wearer. Bandage 520 is illustrated wrapped around and adhered to finger 530. Bandage 520 can be made of generally non-conductive material. Isolated conductive paths 522 and 524 are illustrated providing a path for electrical conduction through bandage 520. In the embodiment of FIG. 5, an adhesive pad 540 is illustrated including conductive material at locations 542 proximate to conductive path 522 and 544 proximate to conductive path 524. Exemplary electrical circuit 550 is illustrated starting in touchscreen surface 510, going through conductive path 522, location 542, finger 530, location 544, conductive path 524, and back into touchscreen surface 510. In one embodiment, the adhesive used to attach the bandage to the skin of the patient can additionally include conductive properties, permitting electrical conduction therethrough. The embodiment of FIG. 5 can be beneficial in that only a small percentage of the material in bandage 520 and pad 540 need be conductive for the embodiment to work as disclosed with a touchscreen device.

Conductive threads, conductive fibers, or conductive can be made of any of a number of conductive materials. Copper, aluminum, or ferrous materials are non-limiting exemplary materials that conduct electricity well and are malleable enough to be used in a flexible bandage.

Conductive threads can be highly conductive, and touchscreen devices only need a small amount of conductivity to sense conduction from one location on the screen to another, so patterns disclosed herein using a blend of conductive and non-conductive threads can include a high percentage of non-conductive threads with only a small percentage of conductive threads. Metallic threads can be expensive relative to a price of normal cloth threads, so such a configuration can incur substantially smaller cost to manufacture as compared to a cloth including most or only metallic threads. In one example, non-conductive threads can make up a majority of the threads in a cloth layer in a conductive bandage as disclosed herein. In another example, non-conductive threads can make up seventy five percent of the threads in a cloth layer in a conductive bandage as disclosed herein. In another example, non-conductive threads can make up ninety percent of the threads in a cloth layer in a conductive bandage as disclosed herein.

In any of the embodiments disclosed herein, silver can be used as all or a portion of the conductive materials used in the bandages. Silver used in dressings has been shown in some studies to aid in the healing process. Silver in the bandages can be used in the cloth layer, the absorbent layer, or both. Silver can be used on an adhesive side of the bandage close to the wound, and another, cheaper conductive material, such as a copper, can be used on the non-adhesive side of the bandage. In such a two material bandage, the threads can be spaced or cross-hatch threaded at 90 degree angles to facilitate electrically conductive contact between the conductive materials, facilitating the touchscreen uses disclosed herein.

Thin strands of silver and/or other conductive materials can be used to make an entire thread for use in a bandage, with a plurality of small strands twisted or braided around each other to make the thread used to weave the cloth of the bandage. A single, larger diameter strand of silver can be used as a thread. A thin strand or a plurality of thin strands can be twisted or braided with other materials to form a composite thread. For example, one silver strand could be braided with a plurality of copper strands. In another example two silver strands could be twisted with cotton fibers to make a thread. A number of different thread configurations are envisioned, and the disclosure is not intended to be limited to the particular embodiments described herein.

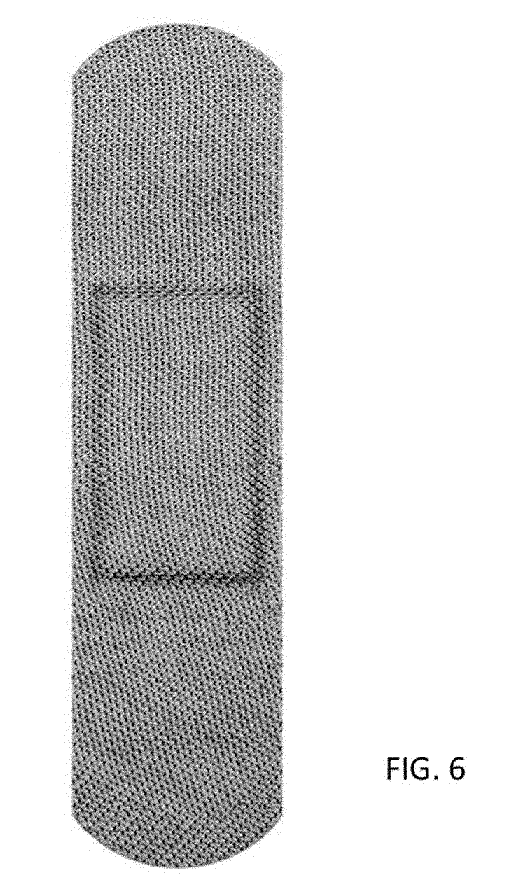

FIG. 6 illustrates through a photographic image a design for a bandage including metallic material in the cloth portion of the bandage, wherein the metallic material is spaced evenly throughout the cloth fibers and is present is sufficient quantity to provide a metallic appearance to the cloth portion of the bandage.

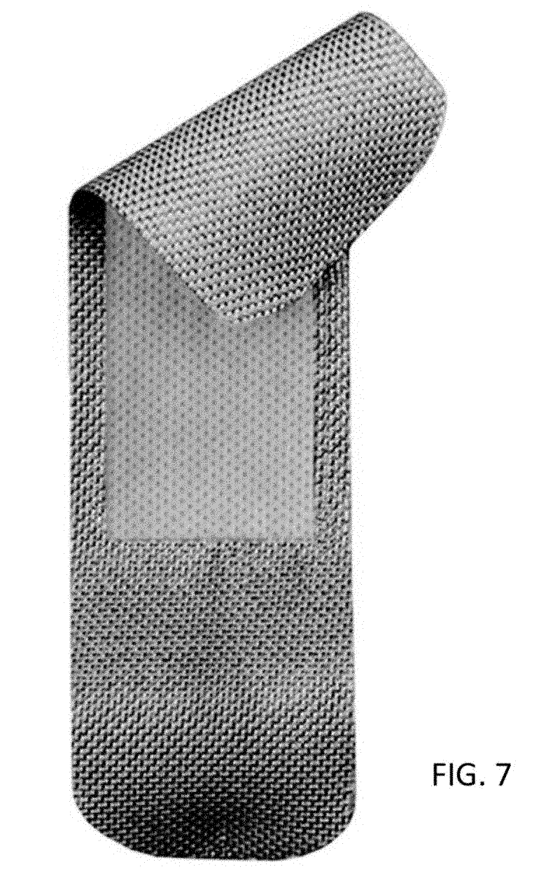

FIG. 7 illustrates through a photographic image the design of FIG. 6.

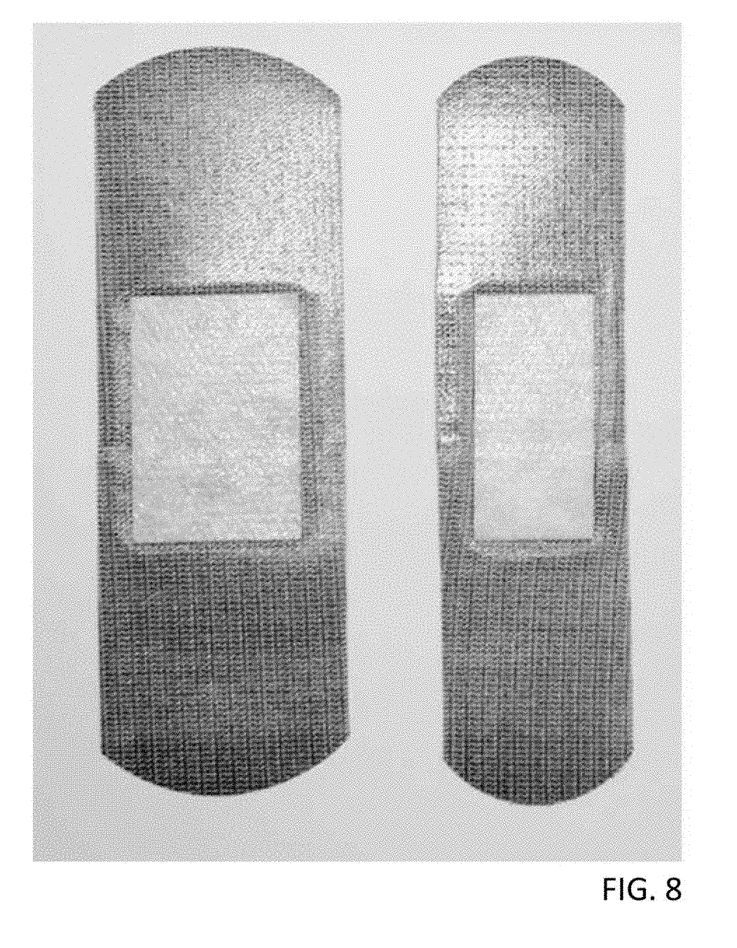

FIG. 8 illustrates through a photographic image alternate configurations of the design of FIG. 6, the configurations being part of the same single design as illustrated in FIG. 6.

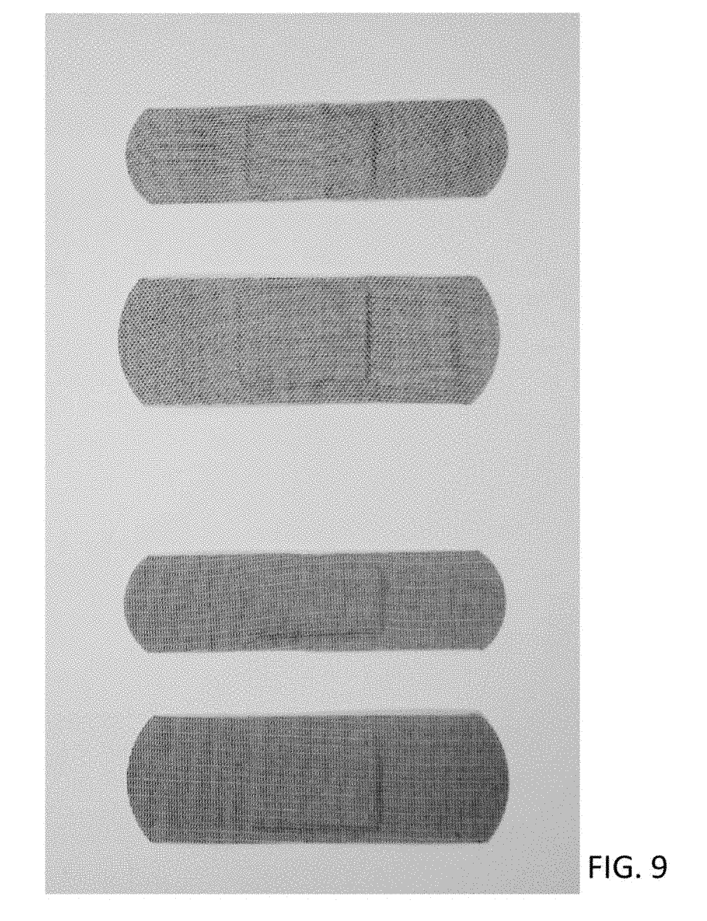

FIG. 9 illustrates through a photographic image alternate configurations of the design of FIG. 6, the configurations being part of the same single design as illustrated in FIG. 6.

A number of alternative embodiments of the disclosed bandage are envisioned. For example, a bandage can include a conductive polymer. Such a conductive polymer, latex, or rubber bandage base layer can include a coated layer of oligodynamic effect coated materials, such as but not limited to silver or copper. The oligodynamic effect is a noted biocidal or anti-microbial effect that occurs in the presence of metals, especially heavy metals, even when the metal is present only in small, visually imperceptible quantities. A bandage such as a conductive polymerized bandage, treated with small or very small amounts of exemplary silver particles, can provide a bandage with inherent anti-microbial properties without the use of anti-biotic creams or lotions.

In another embodiment, a cotton pad or other absorbent pad can include a thin layer of silver or other metal, with that surface exposed to the skin of the user. The layer can be so thin as to still be porous and permitting liquids to reach the pad.

A number of processes are known for providing very small concentrations of silver or other metals upon a surface such as a bandage surface. For example, electroless plating, sputtering, dip coating, metalizing process and nano-particle techniques known in the art can be utilized. In another embodiment, small amounts of silver can be sprayed or infused into the composition used to make the materials in the bandage surface, such that the bandage is formed with a small concentration of silver particles within the bandage material. In another embodiment, small amounts of silver can be sprayed or infused into the adhesive prior to application to the bandage. The silver can exist as small flecks of silver, a thin layer of silver, or as individual silver ions.

The disclosure has described certain preferred embodiments and modifications of those embodiments. Further modifications and alterations may occur to others upon reading and understanding the specification. Therefore, it is intended that the disclosure not be limited to the particular embodiment(s) disclosed as the best mode contemplated for carrying out this disclosure, but that the disclosure will include all embodiments falling within the scope of the appended claims.

What is claimed is:

1. An apparatus comprising a bandage for placement upon skin of a patient and enabling use of a touchscreen device, the apparatus comprising:
the bandage comprising:
adhesive configured to attach the bandage to the skin; and
a polymer-based bandage surface providing electrical conductivity between a plurality of locations on a surface of the bandage, the polymer-based bandage surface comprising one of an anti-static conductive polymer adhesive, a conductive silicone rubber, and a conductive foam material.

2. The apparatus of claim 1, wherein the polymer-based bandage surface comprises the anti-static conductive polymer adhesive.

3. The apparatus of claim 1, wherein the polymer-based bandage surface comprises the conductive silicone rubber.

4. The apparatus of claim 1, wherein the polymer-based bandage surface comprises the conductive foam material.

5. The apparatus of claim 1, wherein the polymer-based bandage surface further comprises a layer of metal particles configured to provide an oligodynamic effect.

6. The apparatus of claim 1, wherein the polymer-based bandage surface further comprises infused metal particles configured to provide an oligodynamic effect.

7. The apparatus of claim 1, wherein the adhesive further comprises infused metal particles configured to provide an oligodynamic effect.

8. The apparatus of claim 1, further comprising an absorbent pad comprising a layer of metal particles configured to provide an oligodynamic effect.

9. The apparatus of claim 1, wherein the polymer-based bandage surface further comprises silver ions configured to provide an oligodynamic effect.

10. The apparatus of claim 1, wherein the adhesive further comprises silver ions configured to provide an oligodynamic effect.

11. The apparatus of claim 1, further comprising an absorbent pad comprising a layer of silver ions configured to provide an oligodynamic effect.

12. An apparatus comprising a bandage for placement upon skin of a patient and enabling use of a touchscreen device, the apparatus comprising:
the bandage comprising:
adhesive configured to attach the bandage to the skin; and
a bandage surface providing electrical conductivity between a plurality of locations on a surface of the bandage, the bandage surface comprising a metal-particle-infused conductive polymer.
